(19) United States
(12) Patent Application Publication
(10) Pub. No.: US 2026/0053774 A1
(43) Pub. Date: Feb. 26, 2026

(54) PARENTERAL COMPOSITIONS OF ZILEUTON AND METHODS OF USE

(71) Applicant: University of Houston System, Houston, TX (US)

(72) Inventors: Vincent Tam, Houston, TX (US); Xinli Liu, Houston, TX (US); Cole Hudson, Houston, TX (US)

(21) Appl. No.: 19/105,589

(22) PCT Filed: Aug. 23, 2023
(86) PCT No.: PCT/US2023/072771

§ 371 Date: Feb. 21, 2025

Related U.S. Application Data
(60) Provisional application No. 63/373,275, filed on Aug. 23, 2022.

(51) Int. Cl. A61K 31/381; A61K 9/51; A61K 31/65; A61K 31/675; A61K 31/7032; A61K 31/7036; A61K 38/12; A61K 39/395; A61P 13/12

(52) U.S. Cl. CPC A61K 31/381; A61K 9/5123; A61K 9/5146; A61K 9/5161; A61K 31/65; A61K 31/675; A61K 31/7032; A61K 31/7036; A61K 38/12; A61K 39/39558; A61P 13/12

(57) ABSTRACT

Provided herein are compositions containing zileuton formulated for parenteral administration and/or extended use. Methods of treatment are directed to subjects who would benefit from extended use of zileuton to provide renal protective effects.

### CROSS-REFERENCE TO RELATED APPLICATIONS

This present patent application claims priority to, and the benefit of, the pending U.S. Provisional Patent Application No. 63/373,275, filed Aug. 23, 2022, the entire content of which is incorporated by reference herein.

### TECHNICAL FIELD

The disclosure relates to parenteral compositions of zileuton and methods of use of these compositions for treatment of disease.

### BACKGROUND

Zileuton is a 5-lipoxygenase inhibitor that inhibits the formation of leukotrienes, such as LTB4, LTC4, LTD4, and LTE4. Zileuton is approved by the FDA (under the trade name Zyflo®) for the prophylaxis and chronic treatment of asthma in adults and children twelve (12) years of age and older. For this indication, zileuton is commercially formulated as tablets for oral administration (e.g., regular, film-coated, or extended release tablets). In addition to the indication of zileuton to treat or prevent asthma, studies support the benefits of zileuton to treat other diseases or conditions.

### SUMMARY

Accordingly, Applicant has recognized a need for a pharmaceutical formulation of zileuton suitable for parenteral route of drug administration (e.g., as an intravenous infusion). These compositions may be used for treating subjects for whom the oral route of administration is not a viable or reliable option or for treating diseases or conditions for which parenteral administration is suitable. The present disclosure provides formulations, compositions, kits, methods of preparation, and methods of treatment with zileuton formulated for parenteral administration. Methods of treating diseases or conditions that would benefit from extended use of zileuton for renal protective effects, e.g., infection and cancer for which nephrotoxicity-inducing treatment is used, are also provided. Through virtual drug screening, additional pharmacological properties of zileuton were developed, which can be exerted through extended use. For example, preliminary studies in experimental animals support the benefits of zileuton to reduce nephrotoxicity associated with selected antibiotics (e.g., polymyxins and aminoglycosides). The subject population that would benefit from the extended use of zileuton includes subjects with acute and serious infections, including critically ill subjects for whom the oral route of administration is not a viable or reliable option, or subjects who have diseases or conditions for treatment of which parenteral administration of zileuton is more suitable than oral administration.

One aspect of the present disclosure provides a pharmaceutical composition containing a therapeutically effective amount of zileuton and a biologically safe excipient formulated for parenteral administration. The pharmaceutical composition can be used for preventing, delaying onset of, or treating acute kidney injury (AKI) or nephrotoxicity. In certain examples, the excipient is polyethylene glycol (PEG). The PEG component can include PEG with molecular weight ranging from about 350 to about 650 Daltons, such as PEG 400, PEG 500, PEG 600, and PEG 400-600. Excipients can include propylene glycol, glycerin, ethanol, sorbitol, or any combinations thereof. In certain examples, the excipient contains ethanol, PEG 400, and water in 1:4:5 or 10%:40%:50% in volume. In certain examples, the excipient contains about 10 vol. % ethanol, about 40 vol. % PEG 400, and about 50 vol. % water. In certain examples, the excipient contains ethanol, PEG 400, and water in 10%:25%:65% in volume. In certain examples, the excipient contains about 10 vol. % ethanol, about 25 vol. % PEG 400, and about 65 vol. % water.

In one aspect, the present disclosure provides a nanoparticle-based composition containing a therapeutically effective amount of zileuton and a nanoparticle, wherein the parenteral composition is formulated for renal protection against a nephrotoxicity-inducing agent. In certain examples, the nanoparticle contains polysaccharide, a pegylated lipid, and/or phosphate counter ions. In certain examples, the nanoparticle contains a mean hydrodynamic diameter ranging from about 50 nanometers (nm) to about 300 nm and near neutral charge. In certain examples, the nanoparticle contains a mean hydrodynamic diameter of about 280 nm and a mean zeta potential of about −3 mV. In certain examples, the nanoparticle contains zileuton in an inner cavity of the nanoparticle. In certain examples, the composition accumulates in proximal tubule epithelia when administered to subjects.

In certain examples, the parental compositions or nanoparticle-based compositions provided herein contain about 1 mg/ml zileuton. In certain examples, the zileuton degrades less than 10% over 140 hours to 150 hours at −20 degrees centigrade (° C.), 4° C., or 22° C. in the pharmaceutical composition.

In certain examples, the pharmaceutical composition is used in preventing, delaying onset of, or otherwise treating AKI or nephrotoxicity in a subject. In certain examples, the AKI or nephrotoxicity is induced by an antibiotic or an anti-cancer drug. Administration of the pharmaceutical composition provided herein can have nephroprotective effect against the nephrotoxicity-inducing agent or the AKI-inducing agent.

In certain examples, the nephrotoxicity-inducing agent administered to the subjects contains an antibiotic or an anti-cancer drug. In certain examples, the antibiotic can be one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug contains ifosfamide, ipilimumab, pembrolizumab, and/or nivolumab. The pharmaceutical composition provided herein can be administered intravenously, intraperitoneally, subcutaneously, intramuscularly, or transdermally to a subject.

Embodiments include methods of preparing a parenteral composition by combining an effective amount of zileuton with a biologically acceptable excipient for parenteral administration. Embodiments also include methods of administering a parenteral composition containing an effective amount of zileuton to a subject. In certain examples, the subject has a severe infection or is critically ill or is otherwise unable to handle an oral formulation.

Embodiments include methods of treating a subject with infection by administering a parenteral composition containing an effective amount of a zileuton-containing composition and an effective amount of a nephrotoxicity-inducing antibiotic. The antibiotics can be one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4.

Embodiments include methods of treating a subject with cancer by administering a parenteral composition containing an effective amount of a zileuton-containing composition and an effective amount of a nephrotoxicity-inducing anticancer drug. The anti-cancer drug can be one or more of ifosfamide, ipilimumab, pembrolizumab, and nivolumab. In certain examples, the effective amount of zileuton can range from about 0.5 mg/kg to about 200 mg/kg. In certain examples, the zileuton-containing composition can be administered intravenously, intraperitoneally, subcutaneously, intramuscularly, or transdermally.

Embodiments further include kits for parenteral administration of zileuton. In certain examples, the kits include an instruction for use and a pharmaceutical composition containing a therapeutically effective amount of zileuton and a biologically acceptable excipient.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to illustrate embodiments of the disclosure more clearly.

FIGS. 1A-1B depicts chromatogram of zileuton (the USP standard). FIG. 1A shows separation of analytes by reverse-phase liquid chromatography and detection based on retention times and mass/charge ratios, respectively. Detection was made using multiple reaction monitoring (MRM) scan type in the positive-ion mode. Zileuton-d4 was used as the internal standard (shown in blue on chromatogram). In FIG. 1B, the linear range of the assay was 0.0625-8 μg/ml in rat serum (r²=0.997).

FIG. 2 is a graphical representation of the solubility of zileuton in various solvents generally regarded as safe (GRAS) by the FDA for parenteral pharmaceutical formulation.

FIG. 3 depicts a ternary diagram of solubility for zileuton (1 mg/ml). Data for an example formulation according to an embodiment is shown by star (65% water, 25% PEG 400, 10% ethanol).

FIG. 4 is a graphical representation of the stability of zileuton in an example formulation (65% water, 25% PEG 400, 10% ethanol).

FIGS. 5A-5D are graphical representations of the pharmacokinetic profiles of zileuton in serum (FIG. 5A: 4 mg/kg, FIG. 5C: 12 mg/kg) and renal tissue (FIG. 5B: 4 mg/kg, FIG. 5D: 12 mg/kg) in rats.

FIG. 6 is a graphical representation of the pharmacokinetics of zileuton at steady state or following single dose administration. To provide steady state, Zileuton (12 mg/kg) in an example formulation (in 65% water, 25% PEG 400, 10% ethanol) was given once daily with for 10 days.

FIG. 7 is a graphical representation of the onset of nephrotoxicity associated with amikacin in rats. Animals in both cohorts received amikacin (AMK) 300 mg/kg daily. Both cohorts (administered or not administered zileuton) were comprised of 10 animals (5 males and 5 females). The endpoint was defined as ≥2× elevation of serum creatinine value from baseline for each animal. p=0.015.

FIGS. 8A-8C depicts histological evidence of renal injury in kidney tissue of rats administered amikacin (FIG. 8A), vehicle (FIG. 8B), or amikacin and zileuton (FIG. 8C). The histology sections were PAS stained and shown in 400×.

FIG. 9 is a graphical representation of the onset of nephrotoxicity associated with polymyxin B in rats. Animals in both cohorts received polymyxin B (PB) 20 mg/kg daily. Both cohorts (first cohort administered PB alone, and second cohort administered PB and zileuton) were comprised of 10 animals (both genders). The polymyxin B alone group consisted of 5 animals receiving the vehicle and 5 animals that received no vehicle. The two control groups were deemed comparable (thus combined for the analysis) as the median time to reach nephrotoxicity was 5 versus 6 days, respectively. The endpoint was defined as ≥2× elevation of serum creatinine value from baseline for each animal. p<0.01.

FIGS. 10A and 10B are graphical representations of the dose-response (reduction in nephrotoxicity) associated with zileuton. FIG. 10A depicts reduction in nephrotoxicity associated with 300 mg/kg amikacin with concomitant zileuton (1 and 4 mg/kg). FIG. 10B depicts reduction in nephrotoxicity associated with 20 mg/kg polymyxin B with concomitant zileuton (4 and 10 mg/kg). Nephrotoxicity was defined as a ≥2× elevation of each animals' baseline serum creatinine over 10 days. N=10 in each group.

FIGS. 11A-11C are graphical representations of the multi-dose safety of zileuton. Zileuton 12 mg/kg in an example formulation (in 65% water, 25% PEG 400, 10% ethanol) was administered to rats once daily for 10 days (day 1-10), and weight (FIG. 11A), serum creatinine (FIG. 11B), and ALT (FIG. 11C) were measured at baseline, day 5, day 10, or day 14 as indicated.

FIG. 12 schematically depicts nanoparticles (NPs) and the dynamic light scatter analysis of NPs showing a monodisperse peak with mean diameter of 280 nm and zeta potential of −3 mV.

FIGS. 13A-13C depicts in vivo imaging of near-infrared dye conjugated AF750-NPs in nude mice. FIG. 13A shows fluorescence imaging of NPs in mice for up to 72 hrs. FIG. 13B shows ex vivo fluorescence imaging of major organs 3 days post IV injection of NPs. LIV: liver, SPL: spleen, NK: normal kidney. FIG. 13C shows multispectral optoacoustic tomography (MOST) imaging of mouse kidneys 3 days after IV AF750-NPs administration. The green color indicates the fluorescent NP. N=3 mice per imaging group.

FIGS. 14A-14D depict fluorescence images (40×) of kidney after 3 days post IV administration of AF750-labeled NPs in mice. Kidneys were stained with anti-mouse megalin antibody and cell nuclei was stained with DAPI (Red: NPs (FIG. 14A). Green: megalin (FIG. 14B), Blue: nuclei (FIG. 14C), and all overlaid in FIG. 14D) (n=3). Specific targeting of the proximal tubular cells of kidney in mice were evaluated.

### DETAILED DESCRIPTION

The description may use the phrases “in certain examples,” “in certain embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

Zileuton contains one weakly acidic hydrogen atom and one asymmetric center and thus gives rise to the formation of salt forms and enantiomers that may be defined, in terms of absolute stereochemistry, as (R)- or (S)- and in terms of their optical rotations as (+) and (−) enantiomers. The present disclosure includes all salt forms and racemic mixtures, optically pure forms and cyclodextrin derived inclusion complex mixtures. The sodium salt of zileuton is commercially available and the optically active (R)- and (S)-isomers may be prepared using known chiral synthons, chiral reagents, or separated into pure enantiomers using other means known in the art. “Stereoisomers” are isomers that differ only in the way the atoms are arranged in space. “Enantiomers” are a pair of stereoisomers that are non-superimposable mirror images of each other. A 1:1 mixture of a pair of enantiomers is called a “racemic” mixture. The term “(+)” is used to designate a racemic mixture where appropriate. The absolute stereochemistry is specified according to the CahnIngold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. It is intended that the compounds described herein include racemates, both E and Z geometric isomers and various pharmaceutically acceptable salt forms. As used, herein, the term “zileuton” encompasses ((±) 1-[1-(1-benzothiophen-2-yl)ethyl]-1-hydroxyurea, the optically pure form of the (S)-enantiomer or (−)-isomer of N-(1-benzo[b]thien-2-ylethyl)-N-hydroxyurea (as described, for example, in U.S. Pat. No. 5,629,337), the optically pure form of (R)-enantiomer or (+)-isomer of N-(1-benzo[b]thien-2-ylethyl)-N-hydroxyurea (as described, for example, in WO 94/26268) and mixtures of the (S)- and (R)-isomers in any ratio between 1:99 and 99:1, and polymorphic forms of zileuton. Certain embodiments include the zileuton salts, such as the sodium salt of zileuton (sodium; 1-[1-(lbenzothiophen-2-yl)ethyl]-1-oxidourea).

As used herein, a “subject” is an animal, such as a mammal, including a primate (such as a human, a non-human primate, e.g., a monkey) and a non-primate (such as a cow, a dog, a horse, a sheep, a rabbit, a cat, a rat, or a mouse). In some aspects of the invention, the subject is a human, such as a human having, suspected of having, or at risk of developing, infection or cancer for which treatment with a nephrotoxicity-inducing drug is indicated. The subject can be an adult subject or a pediatric subject, such as a neonate, an infant, or a child.

As used herein, “treating” a condition or “treatment” of a condition (e.g., nephrotoxicity, renal insufficiency, or AKI associated with use of nephrotoxicity-inducing agent for, e.g., treatment of infection or cancer) refers to a beneficial or desired result, such as reducing at least one sign, symptom, or complication associated with the condition. “Treating” or “treatment” also refers to delaying onset or progression of the condition, or signs, symptoms, or complications associated with the condition in a subject. “Treating” or “treatment” also refers to a preventive or prophylactic treatment, such as prevention of a condition or prevention of at least one sign, symptom, or complication associated with the condition. Accordingly, “treatment” can refer to a reduction in likelihood of developing a disease or associated signs, symptoms, conditions, or complications, or a reduction in severity of a disease or associated signs, symptoms, conditions, or complications relative to a population having the same risk factors and not receiving treatment as described herein. The failure to develop a disease, or a delay in time to develop associated signs, symptoms, conditions, or complications by days, weeks, months, or years is considered effective treatment. Treatment may require administration of more than one dose of the pharmaceutical compositions described elsewhere herein. “Treatment” can also mean prolonging survival as compared to expected survival in the absence of treatment.

The term “reducing,” “reduced,” “decreasing,” “decreased,” or any variation thereof, when used in the claims and/or the specification includes any measurable decrease of one or more components in a mixture to achieve a desired result, such as a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “reduced”, “decreased”, and the like encompass both a partial reduction and a complete reduction compared to a control.

The term “increased,” “increasing,” “increase,” or any variation thereof refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, e.g., an established normal or reference level of the parameter, or an established standard control. Accordingly, the terms “increased” and the like encompass both a partial increase and a significant increase compared to a control.

Zileuton is a commercially available 5-lipoxygenase inhibitor indicated for the treatment of chronic asthma. Compositions and methods containing zileuton for treating diseases or conditions other than asthma are described in WO2020/092016.

Certain embodiments of this disclosure include parenteral compositions for treating bacterial infections or cancer in a mammal containing an effective amount of zileuton or a pharmaceutically acceptable salt thereof and an effective amount of one or more nephrotoxicity- or neurotoxicity-inducing antibiotics or anti-cancer drugs, respectively, and methods of treating infections or cancer in a mammal by administering to the mammal an effective amount of the parenteral composition. Also provided herein are parenteral compositions for treating acute kidney injury or diabetic nephropathy with an effective amount of zileuton or a pharmaceutically acceptable salt thereof, and methods of treating acute kidney injury or diabetic nephropathy in a mammal by parenterally administering to the mammal an effective amount of the composition.

Globally, the prevalence of antimicrobial resistance (AMR) has been increasing for decades. Currently, resistance to first-line antibiotics (such as beta-lactams and fluoroquinolones) is widespread and presenting a major health threat. Last-line antibiotics (such as aminoglycosides and polymyxins) have maintained in vitro activity against multidrug-resistant (MDR) Gram-negative bacteria such as A. baumannii, K. pneumoniae, and Pseudomonas aeruginosa, three “superbugs” listed by the Infectious Disease Society of America as requiring urgent action. However, aminoglycosides and polymyxins are associated with AKI, hindering their clinical usage to treat MDR Gram-negative bacterial infections. The rise of AMR has led to an increased interest in identifying strategies to reduce the renal toxicity of these antibiotics. Due to the low prevalence of resistance in most bacteria and the widespread availability of these antibiotics, attenuation of amikacin and polymyxin B-associated AKI would be a significant and timely accomplishment to combat the AMR crisis. As disclosed herein, inventors have developed the use of zileuton to reduce aminoglycoside and polymyxin-associated nephrotoxicity associated with AKI.

The zileuton composition for parenteral administration provided herein can be prepared by combining a therapeutically effective amount of zileuton with a biologically acceptable excipient (e.g., solvent or nanoparticle). The zileuton composition can contain at least about 1 mg/ml of zileuton.

Provided herein are methods of administering the zileuton composition parenterally to a subject, such as to treat or prevent a disease or a condition. For example, the pharmaceutical composition can be used in preventing, delaying onset of, or treating AKI or nephrotoxicity in a subject. In certain examples, the AKI or nephrotoxicity is induced by an antibiotic or an anti-cancer drug provided herein.

With respect to the compositions and methods for treating bacterial infections, the nephrotoxicity- or neurotoxicity-inducing antibiotics can be one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. The aforementioned composition can further include one or more of amikacin, apramycin, azithromycin, aztreonam, menopenem-vaborbactam, imipenem-relebactam, ceftazidime-avibactam, ceftolozane-tazobactan, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin.

With respect to the compositions and methods for treating cancer, the nephrotoxicity- or neurotoxicity-inducing anticancer drugs can be one or more ifosfamide or an immune checkpoint inhibitor such as for example, ipilimumab, pembrolizumab, or nivolumab; and a pharmaceutically acceptable carrier.

Also provided are methods of treating infection, inflammation, cancer, acute kidney injury, and/or diabetic nephropathy in a subject by administering zileuton or a composition containing zileuton to the subject, and suitable formulation and dosage thereof, including extended use. “Extended use” of zileuton as used herein refers to therapeutic use of a zileuton composition beyond what is originally approved by the FDA, which is for treating or preventing asthma. Extended use of zileuton can be for any disease or condition for which use of zileuton is therapeutically beneficial, including for preventing, delaying onset of, or treating nephrotoxicity or AKI associated with use of nephrotoxicity-inducing agents for infection or cancer treatment. Extended use includes parenteral administration, intravenous administration, intraperitoneal administration, subcutaneous administration, intramuscular administration, transdermal administration, continuous use, and non-continuous use (e.g., periodic, regular, or repeated use) for the duration of time in which the zileuton composition was used (e.g., administered to the subject).

In one aspect, provided herein is a method of treating a subject with infection, by administering parenterally an effective amount of the zileuton composition and an effective amount of a nephrotoxicity-inducing antibiotic selected from the group consisting of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4.

In one aspect, provided herein is a method of treating a subject with cancer, by administering parenterally an effective amount of the zileuton composition provided herein and an effective amount of a nephrotoxicity-inducing anticancer drug, such as ifosfamide, ipilimumab, pembrolizumab and nivolumab.

In certain examples, the effective amount of zileuton is about 0.5-200 mg/kg. Zileuton can be administered by any route suited for clinical purpose, for example intravenously, intraperitoneally, subcutaneously, intramuscularly, or transdermally. The zileuton composition can be administered in a single dose or for multiple dose for an extended time period.

Currently, zileuton is formulated as (regular, film-coated and extended release) tablets for oral administration. However, in target subject population for the extended use of zileuton (e.g., critically ill subjects, subjects with hemodynamic instability, and/or subjects with multidrug resistant bacterial infections for whom polymyxins are used as last resort treatment), the oral route is not viable/reliable for systemic delivery of the active ingredient to achieve the desirable therapeutic effect. Accordingly, provided herein is a pharmaceutical composition formulated for parenteral route of drug administration (e.g., as an intravenous infusion). Also provided herein is a method of preparing the pharmaceutical composition. The parenteral zileuton composition can be prepared by combining a therapeutically effective amount of zileuton with a biologically acceptable excipient.

In some aspects, provided herein is a co-solvent system for parenteral administration of zileuton. Zileuton (MW=236) has a log P value of 0.9 and is practically insoluble in water (<0.5 mg/ml). In certain examples, the co-solvent parenteral formulation for zileuton contains FDA approved excipients. In certain examples, the excipient is polyethylene glycol (PEG). The PEG can range from about 20 vol. % to about 50 vol. % of the excipient. The excipient component can include PEG with molecular weight ranging from about 350 to about 650 Daltons (such as PEG 400, PEG 500, PEG 600, or PEG 400-600), propylene glycol, glycerin, ethanol, sorbitol, or any combinations thereof. In certain examples, the excipient contains ethanol, PEG 400, and water in 1:4:5 or 10%:40%:50% in volume. In certain examples, the excipient contains about 10 vol. % ethanol, about 40 vol. % PEG 400, and about 50 vol. % water. In certain examples, the excipient contains ethanol, PEG 400, and water in 10%:25%:65% in volume. In certain examples, the excipient contains about 10 vol. % ethanol, about 25 vol. % PEG 400, and about 65 vol. % water. The formulation can generate 1-10 mg/ml zileuton injectable solution to support preclinical or clinical investigations. The target dose of zileuton can be determined based on various clinical factors (e.g., subject weight). The injection volume can be adjusted accordingly. For instance, in the rodent model, 1 mg/ml zileuton with a volume ranging from 1 ml to 3 ml can be given intraperitoneally.

Zileuton can first be dissolved in ethanol as a solution of 10 mg/ml and stored in −20° C. Immediately prior to dosing, 0.1 ml of the ethanol solution can be diluted 1:9 with PEG 400:water (4:5 co-solvent) to provide a 1 mg/ml final concentration of zileuton. Based on the target dose (e.g., based on subject weight), the injection volume can be adjusted accordingly when given intraperitoneally or via any suitable parenteral route, including but not limited to intraperitoneally, subcutaneously, intramuscularly, or transdermally. Zileuton can also be prepared by dissolving in a solvent containing ethanol, PEG 400, and water in a ratio of 10%:25%:65%.

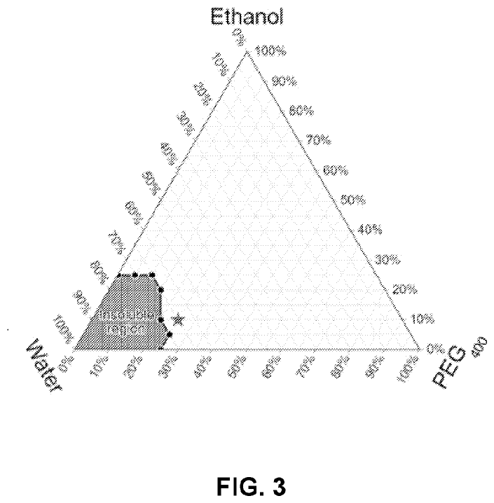

In certain examples, an aqueous composition is provided for parenteral administration containing a therapeutically effective amount of zileuton and a biologically safe excipient, for renal protection against a nephrotoxicity-inducing agent. In certain examples, the excipient is polyethylene glycol, PEG 400-600, propylene glycol, glycerin, ethanol, sorbitol, triacetin, polyoxyethylated glycerides, polyoxyethylated oleic glycerides, hyroxypropyl-beta-cyclodextrin, a surfactant (e.g. hydroxypropylcellulose 20, polysorbate 80, polysorbate 20, sorbitan monooleate NF, polyoxyl 40 hydrogenated castor oil, polyoxyl hydroxystearates), or combinations of any thereof. Any combination of the excipients in any ratio can be used, optionally in combination with water. For example, in certain examples, the excipient is ethanol, PEG 400, and water in 1:4:5 or 10%:25%:65% in volume or in any combination presented by the ratio of ethanol, PEG 400, and water outside the insoluble region of FIG. 3.

“Therapeutically effective amount” or “effective amount” refers to that amount of an agent (e.g., zileuton) effective to produce the intended pharmacological, therapeutic or preventive result, e.g. renal protective effect against nephrotoxicity-inducing agents. An effective amount also may include an amount effective to inhibit or reduce signs or symptoms associated with nephrotoxicity (e.g., decrease in urine output, decrease in creatinine clearance). For example, if a given clinical treatment is considered effective when there is at least a 10% improvement (e.g., increase or decrease that relates to improvement of the disease, condition, or associated signs or symptoms) in a measurable parameter associated with a disease or disorder, a therapeutically effective amount of a drug for the treatment of that disease or disorder is the amount necessary to obtain at least a 10% improvement (e.g., by at least 10%, 15%, 20%, 25%, 30%, 35%, 40%, 45%, 50%, 55%, 60%, 65%, 70%, 75%, 80%, 85%, 90%, or 95%) in that parameter.

The zileuton formulation can be stable. For example, the parenteral zileuton composition degrades less than 10% over 140 hours to 150 hours at −20° C., 4° C., or 22° C.

In some aspects, provided herein is a stable formulation of zileuton for parenteral administration. The stable parenteral formulation of zileuton can include biocompatible nanoparticle compositions that are selectively taken up by renal proximal tubular cells, resulting in an enhanced uptake of renal protectant at the site of renal injury.

Nanoparticles are ultrafine particles that generally have at least one dimension of less than 100 nm in size. Nanoparticles made of biocompatible and biodegradable materials can offer many pharmacokinetic, efficacy, and safety benefits when they are used as drug formulations. Various therapeutic and diagnostic agents can be encapsulated in nanoparticles for targeted delivery. The property and in vivo performance of nanoparticles as drug delivery vehicles are crucially dependent on the nature of biomaterial used, size, surface charge, rigidity, permeability, solubility, stability, etc.

There are two ways through which nanostructures deliver drugs: passive and self-delivery, both of which are within the scope of the present disclosure. In the former, drugs are incorporated in the inner cavity of the structure mainly via the hydrophobic effect. When the nanostructure materials are targeted to specific sites, the intended amount of the drug is released because of the low content of the drugs which is encapsulated in a hydrophobic environment. Conversely, in the latter, the drugs intended for release are directly conjugated to the carrier nanostructure material for easy delivery. In this approach, the timing of release is crucial as the drug will not reach the target site if it dissociates from the carrier very quickly, and conversely, its bioactivity and efficacy will be decreased if it is released from its nanocarrier very slowly. Targeting of drugs is another significant aspect that uses nanomaterials or nanoformulations as the drug delivery systems and, is classified into active and passive. In active targeting, moieties, such as antibodies and peptides are coupled with drug delivery system to anchor them to the receptor structures expressed at the target site. In passive targeting, the prepared drug carrier complex circulates through the bloodstream and is driven to the target site by affinity or binding influenced by properties like pH, temperature, molecular site and shape. The main targets in the body are the receptors on cell membranes, lipid components of the cell membrane and antigens or proteins on the cell surfaces.

In one aspect, provided herein is a nanoparticle-based composition containing therapeutically effective amount of zileuton and a nanoparticle, formulated for parenteral administration for, e.g., renal protection against a nephrotoxicity-inducing agent. The nanoparticle contains polysaccharide, a pegylated lipid, and/or phosphate counter ions.

The size of nanoparticle can be large enough not to easily cross the glomerular filtration barriers and be excreted in the urine (e.g., ≥6 nm), and not too big to be cleared by organs of the reticuloendothelial system (RES) (e.g., ≤500 nm). In certain examples, the nanoparticle contains a mean hydrodynamic diameter of about 50-300 nm (e.g., about 50 nm, 60 nm, 70 nm, 80 nm, 90 nm, 100 nm, 150 nm, 200 nm, 250 nm, 300 nm, 50-100 nm, 100-150 nm, 150-200 nm, 200-250 nm, 250-300 nm) and near neutral charge. In certain examples, the nanoparticle contains a mean hydrodynamic diameter of about 280 nm and a mean zeta potential of about −3 mV. In certain examples, the composition accumulates in proximal tubule epithelia when administered to subjects. In certain examples, zileuton is directly or externally conjugated to nanoparticles, which can deliver zileuton to the target tissue, e.g., proximal tubule epithelia of the kidney (e.g., active targeting). In certain examples, zileuton is incorporated in the inner cavity of nanoparticles, which can deliver zileuton to the target tissue, e.g., proximal tubule epithelia of the kidney (e.g., passive targeting).

A subject (e.g., a human subject, e.g., a patient) can be administered a therapeutic amount of zileuton composition. The therapeutic amount can be, e.g., 0.05-50 mg/kg body weight. In certain examples, the therapeutic amount of zileuton delivered as a parenteral formulation is from about 1 mg/kg to 12 mg/kg, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, or about 12 mg/kg. In certain examples, a single dose administration of a pharmaceutical composition containing 4 mg/kg zileuton results in a serum concentration of about 880 mg in a human subject. In certain examples, a single administration of a pharmaceutical composition containing 12 mg/kg zileuton results in a serum concentration of about 3000 mg in a human subject.

The zileuton composition of the present disclosure can be administered to a subject via any methods or routes that is suited for the subject. For example, the zileuton composition can be administered via parenteral routes, including intracranial (e.g., intraventricular, intraparenchymal, and intrathecal), intraperitoneal, intravenous (e.g., injection, infusion), intramuscular, subcutaneous, transdermal, airway (aerosol), nasal, ocular, and topical (including buccal and sublingual) administration. Zileuton compositions can be formulated for delivery to a target organ, e.g., to the kidney. In certain examples, the zileuton composition is administered to promote deposition substantially in the kidney, e.g. the proximal tubule.

In certain embodiments, the zileuton composition is taken up in one or more tissues or cell types in the renal system including, but not limited to, tubular epithelia, macula densa, glomerular endothelia, podocytes, Mesangial cells, glomerular basement membrane cells, parietal epithelia. In specific embodiments, the zileuton composition accumulates in tubular epithelia of the kidney, e.g., proximal tubular epithelia.

In certain examples, the administration is repeated, for example, on a regular basis, such as, daily, biweekly (i.e., every two weeks) for one month, two months, three months, four months, six months or longer. After an initial treatment regimen, the treatments can be administered on a less frequent basis. F or example, after administration biweekly for three months, administration can be repeated once per month, for six months or a year or longer.

In certain examples, the parenteral zileuton composition is administered in two or more doses. In certain examples, the number or amount of subsequent doses is dependent on the achievement of a desired effect, e.g., to inhibit or reduce kidney damage associated with co-administration of nephrotoxicity-inducing agents, e.g., reduction or prevention of one or more symptoms associated with acute kidney injury.

In certain examples, the parenteral zileuton composition is administered according to a schedule. For example, the parenteral zileuton composition may be administered once per week, twice per week, three times per week, four times per week, or five times per week. In certain examples, the schedule involves regularly spaced administrations, e.g., hourly, every four hours, every six hours, every eight hours, every twelve hours, daily, every 2 days, every 3 days, every 4 days, every 5 days, weekly, biweekly, or monthly. In certain examples, the parenteral zileuton composition is administered at the frequency required to achieve a desired effect.

In certain examples, the schedule involves closely spaced administrations followed by a longer period of time during which the agent is not administered. For example, the schedule may involve an initial set of doses that are administered in a relatively short period of time (e.g., about every 6 hours, about every 12 hours, about every 24 hours, about every 48 hours, or about every 72 hours) followed by a longer time period (e.g., about 1 week, about 2 weeks, about 3 weeks, about 4 weeks, about 5 weeks, about 6 weeks, about 7 weeks, or about 8 weeks) during which the parenteral zileuton composition is not administered. In certain examples, the parenteral zileuton composition is initially administered hourly and is later administered at a longer interval (e.g., daily, weekly, biweekly, or monthly). In certain examples, the parenteral zileuton composition is initially administered daily and is later administered at a longer interval (e.g., weekly, biweekly, or monthly). In certain embodiments, the longer interval increases over time or is determined based on the achievement of a desired effect. In the course of zileuton administration, different routes of administration can be employed (e.g., intravenous administration in the initial administration, followed by subcutaneous administration in the second administration).

Before administration of a full dose of the zileuton composition, subjects can be administered a smaller dose, such as a 5% infusion dose, and monitored for adverse effects, such as an allergic reaction, or for elevated lipid levels or blood pressure. In another example, the subject can be monitored for unwanted effects.

The parental formulation of zileuton provided herein would be advantageous for a subject who would benefit for the extended use of zileuton. Extended use of zileuton would benefit subjects with acute and serious infections. There are very limited therapeutic options for these subjects due to multidrug resistance, and thus polymyxins or aminoglycosides are used as last resort treatment. The pharmaceutical formulation provided herein, which is suitable for parenteral route of drug administration (e.g., as an intravenous infusion) are highly desirable for these subjects, because some or many of them are critically ill and hospitalized (many are admitted to intensive care units), and under these dire circumstances, the oral route is not reliable or feasible for systemic delivery of zileuton to achieve the desirable (renal protective) effect. In certain examples, the accumulation of zileuton in kidney (e.g., proximal tubule epithelia) is increased by parenteral administration of the parenteral zileuton composition by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more as compared to oral administration of the same or equivalent zileuton composition. In certain examples, renal damage or associated symptoms are reduced by parenteral administration of the parenteral zileuton composition by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to oral administration of the same or equivalent zileuton composition.

The following examples are provided to illustrate further aspects associated with the present disclosure, but should not be construed as limiting the scope thereof. Unless otherwise noted, all parts and percentages are by dry weight.

### EXAMPLES

### Example 1: Development of Co-Solvent Parenteral Formulation of Zileuton

Zileuton (MW=236) has a log P value of 0.9 and is practically insoluble in water (<0.5 mg/ml). It is commercially formulated as tablets for oral administration. Here, a co-solvent parenteral formulation for zileuton was developed using FDA approved excipients:ethanol, PEG 400, and water. In an example, the co-solvents are present in the ratio of 1:4:5 or 10%:25%:65% in volume. The formulation can generate 1-10 mg/ml zileuton injectable solution to support preclinical and clinical uses. For example, Zileuton can first be dissolved in ethanol as a solution of 10 mg/ml and stored in −20° C. Immediately prior to dosing, 0.1 ml of the ethanol solution can be diluted 1:9 with PEG 400:water (4:5 co-solvent) to provide a 1 mg/ml final concentration of zileuton. A zileuton solution (1 mg/ml) was prepared for testing in a rodent model. Based on the target dose (i.e., animal weight), the injection volume can be adjusted accordingly and has ranged from 1 ml to 3 ml given intraperitoneally.

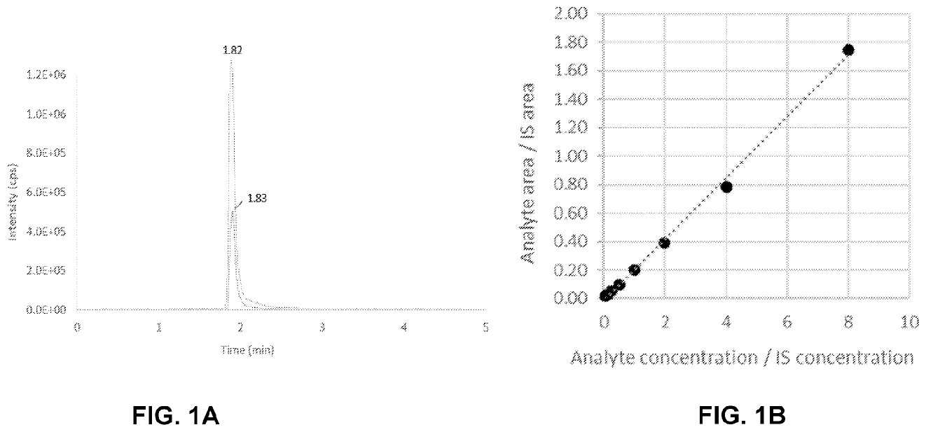

As shown in FIGS. 1A-1B, liquid chromatography-based (LC-MS/MS) assay to quantify zileuton in aqueous medium and biological samples (e.g., serum, cell lysate and kidney tissue homogenate) was developed.

The stable aqueous-based liquid formulation of zileuton is further refined and optimized, using FDA-approved water miscible solubilizing solvents, such as polyethylene glycol (PEG 400-600), propylene glycol, glycerin, ethanol, sorbitol, and combinations thereof. A Central Composite Design (CCD), a widely used approach for systematic design of formulation experiments (Aziz et al. 2018 Curr Drug Deliv 15:1330-42; Hassan et al. 2021 Molecules 26) is employed to optimize the composition of co-solvents required to solubilize zileuton (10-100 mg) with minimum co-solvents. The formulation candidates are screened based on maximum drug solubility, stability, and blood compatibility. The top three (3) most solubilizing excipients in a co-solvent system is further refined based on a three-factor, five-level rotatable CCD matrix with 20 compositions representing 6 center points, 6 axial points, and 8 edge points of surface designs using the Design-Expert® software.

As shown in FIG. 1A, the analytes were separated by reverse-phase liquid chromatography and detected based on retention times and mass/charge ratios, using multiple reaction monitoring (MRM) scan type in the positive-ion mode. Zileuton-d4 was used as the internal standard (shown in blue on chromatogram). As shown in FIG. 1B, the linear range of the assay was 0.0625-8 μg/ml zileuton in rat serum (r²=0.997). All co-solvent formulation compositions are tested for zileuton content by LC-MS/MS (see FIGS. 1A-1B), and for color, appearance (e.g., precipitation and clarity), pH, in vitro hemolytic potential, and stability at different storage temperature (e.g., 4, 25, 37, 60° C.).

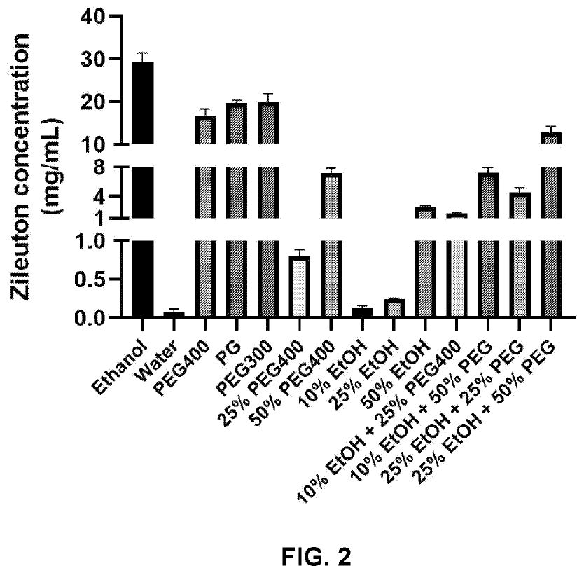

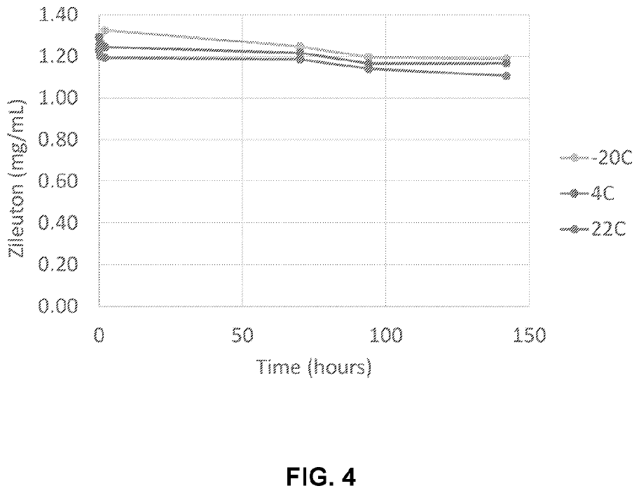

FIG. 2 depicts solubility of zileuton in various solvents generally regarded as safe (GRAS) by the FDA for parenteral pharmaceutical formulation. FIG. 3 presents a ternary diagram of solubility for zileuton (1 mg/ml). As shown in FIGS. 2 and 3, zileuton is soluble in ethanol or PEG 400, and much less soluble in water. Formulation of zileuton can be adjusted by combining ethanol, PEG400, and/or water as solvents. In FIG. 3, Data for an example formulation according to an embodiment is shown by star (65% water, 25% PEG 400, 10% ethanol). As shown in FIG. 4. the example zileuton formulation (65% water, 25% PEG 400, 10% ethanol) is stable for over 142 hours at −20, 4, and 22° C., showing no significant degradation.

### Example 2: In Vivo Evaluation of Co-Solvent Parenteral Formulation of Zileuton

Co-solvent formulations that are safe, non-hemolytic, and effective for parenteral administration are developed as follows.

Up to five (5) top performing co-solvent formulations identified in Example 1 are further evaluated for systemic pharmacokinetics and biodistribution of zileuton after intravenous administration of the optimized formulation in a rodent model. Groups of ten (10) rats (n=5 per gender) are given escalating doses of a single intravenous injection of zileuton (USP). Specific doses to be used is guided by pilot tolerance studies. Blood samples are collected serially, urine/bile samples collected continuously in aliquots, and the kidneys are harvested at selected time points (e.g., 3, 6, 12, 24 h) after an IV injection of zileuton. Zileuton concentrations in these samples are determined using a validated LC-MS/MS assay (as shown in FIGS. 1A-1B). Based on these concentrations, standard pharmacokinetic parameters (e.g., volume of distribution, clearance, etc.) are determined and correlated to the clinical equivalent doses in humans. Special emphasis is paid to bio-distribution profiles to assess renal residence time and metabolic pathway(s) to evaluate drug-drug interactions. The steady state pharmacokinetics of zileuton is also examined after multiple doses.

To ascertain inhibition of in vivo antibiotic uptake by parenteral administration of zileuton, groups of ten (10) rats (n=5 per gender) is given a single intravenous injection of polymyxin B (USP), with or without the co-administration of zileuton 30 mins prior. Specific drug doses to be used mimics clinically relevant dosing in humans. Blood samples are collected serially and the kidneys are harvested at selected time points (e.g., 3, 6, 24 h) to determine polymyxin B concentrations in the blood or tissue, as described previously (see, e.g., Manchadani et al. 2017 Antimicrob Agents Chemother 61:e02391-6). A reduction of >50% polymyxin B concentrations in renal tissue in zileuton-administered animals as compared to a control without zileuton administration is considered a desirable therapeutic effect of zileuton. Similar studies are performed with amikacin (USP).

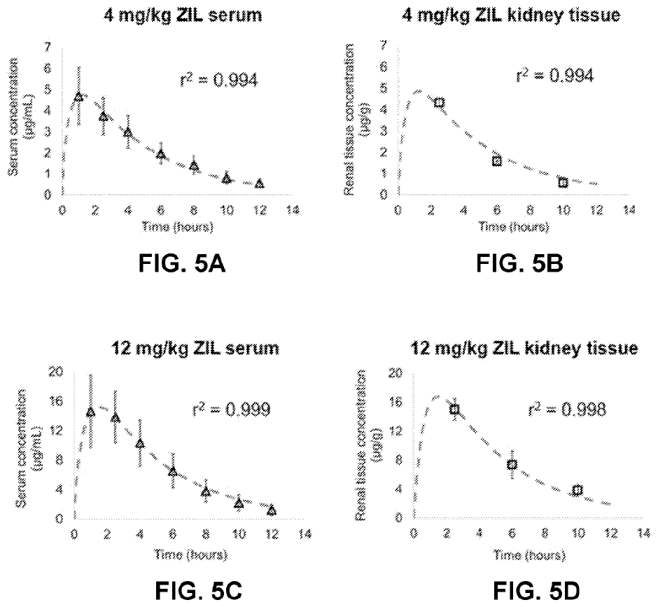

FIGS. 5A-5D depicts pharmacokinetic profiles of zileuton in serum (FIG. 5A: 4 mg/kg, FIG. 5C: 12 mg/kg) and renal tissue (FIG. 5B: 4 mg/kg, FIG. 5D: 12 mg/kg) in rats following single dose administration. Based on proportional serum area under the curve (AUC), the dosing exposure of 4 mg/kg is equivalent to a human dose of approximately 880 mg, and 12 mg/kg is equivalent to a human dose of approximately 3000 mg.

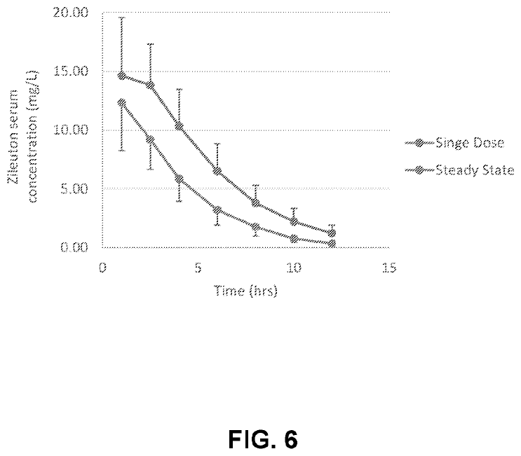

Pharmacokinetics of zileuton at steady state was investigated. To provide steady state, Zileuton (12 mg/kg) in an example formulation (in 65% water, 25% PEG 400, 10% ethanol) was given once daily with for 10 days. As shown in FIG. 6, compared to the first dose, no significant increase in systemic exposure was observed at steady state.

### Example 3: Zileuton Ameliorates Aminoglycoside and Polymyxin-Associated Acute Kidney Injury In Vivo

The effect of zileuton in ameliorating antibiotics (e.g., amikacin, polymyxin B) was studied in rats. As shown in FIGS. 2 and 3, the aqueous solubility of zileuton is poor (0.14 mg/mL). For initial testing against amikacin, zileuton was first dissolved in DMSO (5 mg/mL) and diluted 5× with phosphate buffered saline prior to administering. For subsequent testing against polymyxin B, a zileuton formulation was developed to facilitate administration of a higher dose. A ternary solvent system consisting of 65% water, 25% PEG 400, and 10% ethanol was used to solubilize zileuton to 1 mg/mL.

The zileuton formulation was well-tolerated by the animals. No gross irritation or evidence of drug precipitation was observed in the intraperitoneal cavity following euthanasia. Additionally, absorbance of zileuton at 230 nm was used to confirm stability in the formulation for at least 24 hours at room temperature.

The concentration-time profiles of both doses were reasonably characterized (r²>0.99). The AUC_{0-∞} observed with 4 mg/kg and 10 mg/kg zileuton were found to be 27.2 and 93.5 mg*h/L, respectively. The systemic exposure (mean AUC) following administration of zileuton 600 mg was reported to be 19.2 mg·h/L (package insert—ZYFLO®). Based on proportional AUC, the equivalent doses in humans would be 850 mg and 2,922 mg daily, respectively.

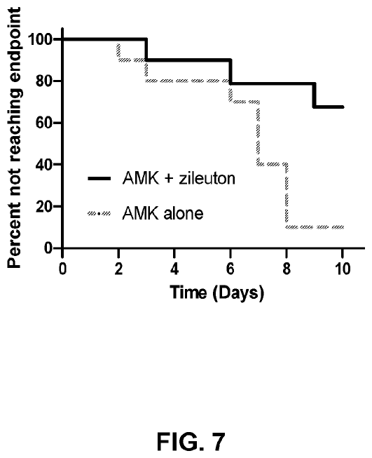

In the amikacin only group, 90% of the animals developed nephrotoxicity within 10 days. The median time to develop nephrotoxicity was 7 days. As shown in FIG. 7, Zileuton (4 mg/kg) delivered prior to amikacin administration significantly delayed the onset of nephrotoxicity (p=0.015). In this group, only 30% of the animals developed nephrotoxicity.

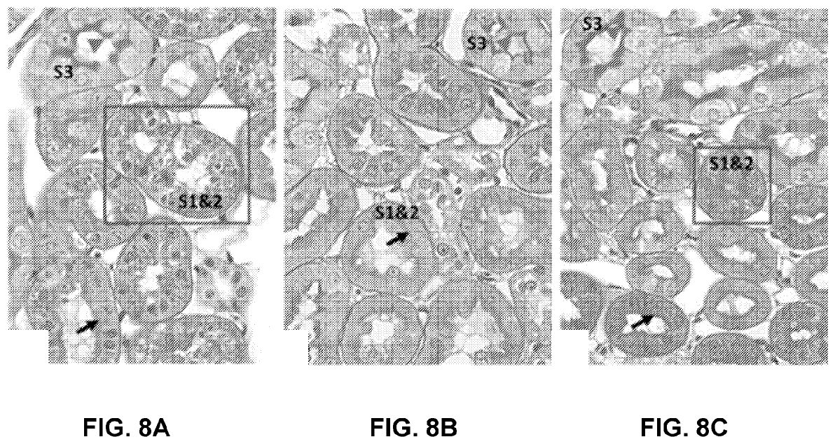

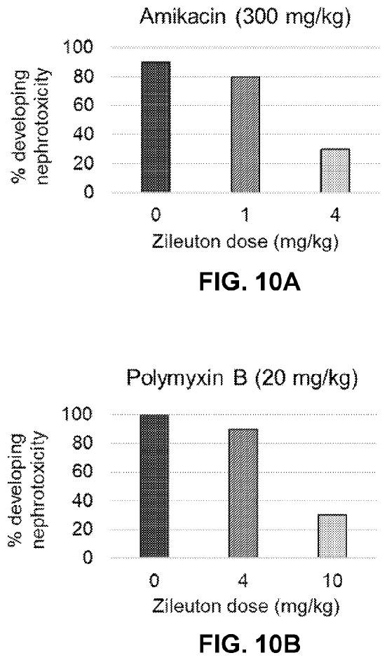

Additionally, as shown in FIG. 8A, histological analysis of kidney sections confirmed significant injury to the proximal tubules in animals treated with amikacin alone. Marked cytoplasmic inclusions, tubular cell injury, loss of brush border (square), involving the S1 and S2 portions of proximal tubules were observed. Preserved brush border in less affected proximal tubular profile (FIG. 8A, arrow). The S3 portion of proximal tubules was mostly intact (S3), showing prominent brush border (FIG. 8A, green arrowhead). As shown in FIG. 8B, in vehicle-treated animals, all portions (FIG. 8B, S1&2, and 3) of proximal tubules were intact without significant changes. The brush border was intact (FIG. 8B, arrow and arrowhead). As shown in FIG. 8C, in animals treated with amikacin and zileuton, less injury was observed as compared with animals treated with amikacin alone. Mild cytoplasmic inclusions and vacuolization involving few S1&2 profiles were observed (FIG. 8C, square). Most tubular profiles were intact with preservation of brush border for both S1&2 (FIG. 8C, arrow) and S3 portion (FIG. 8C, arrowhead). In sum, zileuton considerably reduced kidney injury with only mild damage observed in the S1 and S2 portions of the proximal tubules. As shown in FIG. 10A, the percentage of animals developing amikacin-associated nephrotoxicity reduced in dose-dependent matter by co-administration of zileuton.

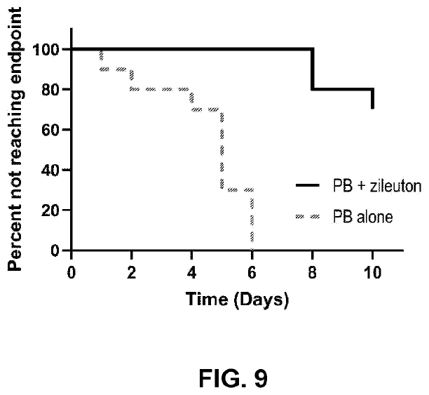

As shown in FIG. 9, in the polymyxin B only group, 100% of the animals developed nephrotoxicity within 10 days. The median time to develop nephrotoxicity was 5 days. Zileuton did not significantly delay nephrotoxicity associated with polymyxin B at 4 mg/kg. As shown in FIG. 9, with a higher dose (10 mg/kg), zileuton significantly delayed the onset of nephrotoxicity (p<0.001). As shown in FIG. 10B, the percentage of animals developing amikacin-associated nephrotoxicity reduced in dose-dependent matter by co-administration of zileuton. Only 30% of animals in the group receiving concomitant zileuton developed nephrotoxicity within 10 days.

Dose-limiting nephrotoxicity is the major barrier preventing the optimal clinical use of aminoglycosides and polymyxins. The mechanism of toxicity is widely attributed to reabsorption of these antibiotics from glomerular filtrate by proximal tubule cells via megalin/PEPT2-mediated transport. The exact intracellular pathway is not completely understood, but once inside renal cells, aminoglycosides/polymyxins accumulate to induce mitochondrial dysfunction, reactive oxygen species (ROS) generation, and caspase activation, resulting in apoptosis. This ultimately leads to major degeneration in the kidney cortex resulting in loss of brush border, increased vascular resistance, and reduced renal blood flow.

Two major therapeutic approaches have been attempted to attenuate nephrotoxicity: (a) reduce antibiotic uptake into proximal tubule cells, or (b) reduce cellular injury following uptake. Reducing drug uptake is a promising approach to attenuate AKI. Anti-inflammatories, mitochondria-acting agents, and antioxidants have also shown potential to reduce cellular injury following aminoglycoside/polymyxin uptake. In contrast to drug uptake inhibition, anti-inflammatories attenuate nephrotoxicity by improving renal perfusion, reducing histological abnormalities, and decreasing the migration of inflammatory cells.

To deliver zileuton parenterally, the present disclosure provides ternary co-solvent formulations of zileuton to overcome its poor solubility in water. The co-solvent formulation achieved a greater than 20-fold increase in aqueous solubility with no apparent side effects. Of note, zileuton had no intrinsic antibacterial activity and it did not interfere with in vitro antibacterial activity or in vivo efficacy of the polymyxins. The data demonstrate zileuton's ability to attenuate nephrotoxicity associated with aminoglycosides and polymyxins in vivo. The onset of nephrotoxicity associated with amikacin and polymyxin B was significantly delayed by concomitant zileuton administration. Furthermore, the overall rates of nephrotoxicity were reduced from 90% (amikacin) and 100% (polymyxin B) to 30% with adjuvant zileuton. Additionally, histopathology confirmed reduced amikacin-associated renal injury in animals receiving zileuton. Specifically, amikacin alone resulted in significant tubular cell injury and loss of brush border, while amikacin given in combination with zileuton resulted in mostly intact tubular profiles and preservation of the brush border. A trend in dose-response was observed for both antibiotics.

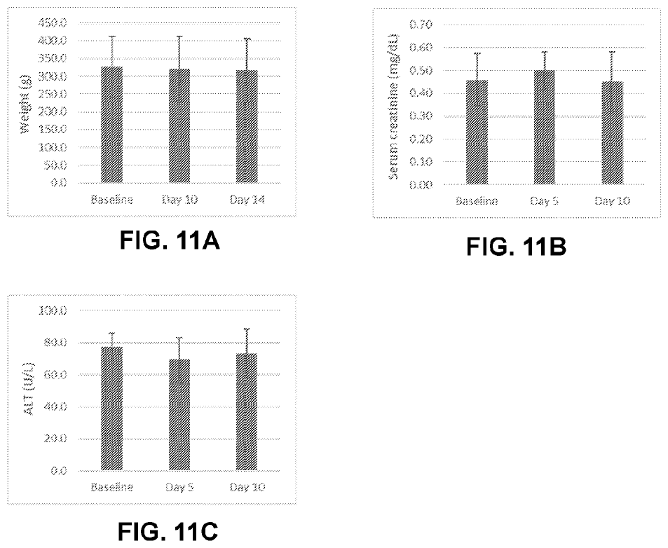

Furthermore, the multi-dose safety of zileuton was confirmed. Zileuton 12 mg/kg was given once daily as the prototype formulation for 10 days (alone without nephrotoxic antibiotics). FIGS. 11A-11C are graphical representations of the multi-dose safety of zileuton. Zileuton 12 mg/kg in an example formulation (in 65% water, 25% PEG 400, 10% ethanol) was administered to rats once daily for 10 days (day 1-10), and weight (FIG. 11A), serum creatinine (FIG. 11B), and ALT (FIG. 11C) were measured at baseline, day 5, day 10, or day 14 as indicated. Compared to baseline, there was no significant change in serum ALT (hepatocellular toxicity marker), serum creatinine (renal function marker), and body weight as observed.

### Materials and Methods

Chemicals and Reagents. Amikacin sulfate (USP) was purchased from Sagent Pharmaceuticals (Schaumburg, Illinois, USA). Polymyxin B (USP) was obtained from Auromedics Pharma (East Windsor, New Jersey, USA). Sterile water and saline were obtained from Covetrus (Portland, Maine, USA). Polyethylene glycol 400 was purchased from Spectrum Chemical (New Brunswick, New Jersey, USA). Ethyl alcohol was obtained from Sigma-Aldrich (St. Louis, Missouri, USA). Zileuton powder (USP) was purchased from Supelco (Bellefonte, Pennsylvania, USA). Piccolo comprehensive metabolic panels were obtained from Abaxis (Union City, California, USA).

Animals. Sprague-Dawley male (weight 325-350 g) and female (weight 225-250 g) rats were obtained from Envigo (Indianapolis, Indiana, USA). Animals were kept on a 12-hour light/dark cycle and received food and water ad libitum. All protocols were approved by the Institutional Animal Care and Use Committee of the University of Houston.

Rat Model of Acute Kidney Injury. Each experimental group was comprised of 10 rats (5 males and 5 females). Animals were administered an antibiotic by subcutaneous injection as previously described (Chan K, et al. Antimicrob Agents Chemother 2020; 64: e00859-20; Manchandani P et al. Antimicrob Agents Chemother 2017; 61: e02391-16). Zileuton (4 mg/kg and 10 mg/kg once daily) was given intraperitoneally, 15 minutes prior to antibiotic administration. Blood samples (approximately 200 μL) were collected at baseline and daily from the tail tip, allowed to clot, and centrifuged at 10,000×G for 10 minutes. The serum was collected and assayed for creatinine concentration by the Piccolo Xpress Chemistry Analyzer (Abaxis, Union City, CA, USA). The endpoint was defined as ≥2× elevation of the baseline serum creatinine value for each animal. The onset of nephrotoxicity in different treatment cohorts was compared using a time-to-event (e.g., Kaplan-Meier) analysis and log rank test. Right censoring was used if the study endpoint was not directly observed within the study timeframe. A p value of <0.05 was considered significant. Kidneys were collected from selected animals for histological examination to characterize the renal injury observed.

As reference controls, amikacin and polymyxin B were given subcutaneously once daily over 10 days. Selection of the amikacin (300 mg/kg) and polymyxin B (20 mg/kg) dose was based on prior pharmacokinetic studies to achieve a total daily drug exposure comparable to that in adults after standard doses.

Kidney histological examination. Kidney sections were examined by light microscopy to assess the extent of kidney injury associated with amikacin exposure. Animals were administered either amikacin only, amikacin and zileuton, or the formulation vehicle only for 3 days. To preserve the kidney tissue, whole-body perfusion-fixation with 10% formalin was conducted. The animals were anesthetized with ketamine/xylazine and perfused with phosphate-buffered saline intracardially using a hydraulic pump (set to 20 mL/min) to flush out the blood. The body was then perfused with 10% formalin for approximately 20 minutes. Both kidneys were removed, fixed in 10% formalin, and stored at 4° C. until analysis. Periodic acid-Schiff reagent was used to stain 1 mm thick sections of the kidneys which were then examined under a light microscope.

Pharmacokinetic studies. Three male and three female rats were administered zileuton (a single dose of 4 and 10 mg/kg) intraperitoneally and serial blood samples (approximately 100 μL each) were taken over 12 hours from the tail tip. The blood samples were allowed to clot at room temperature, centrifuged at 10,000×G for 10 minutes, the serum was collected and stored at −20° C. until analysis. Following quantification, the serum concentrations of each time point were averaged, and the time-profile was characterized using a 1-compartment model with first order absorption in ADAPT 5 (University of Southern California, Los Angeles, CA, USA). AUC_{0-∞} was derived by dividing the dose by the best-fit clearance estimate.

Drug assay. An ultra-performance liquid chromatography tandem mass spectrometry (LC-MS/MS) method was adapted from previous publications for quantification of zileuton in human plasma (Pian Pet al. Journal of Chromatography 2013; 937: 79-83; Armoudjian Y et al. Journal of Pharmaceutical Innovation 2020; 15: 581-90.). Chromatographic separation of zileuton and zileuton-D4 (internal standard) was achieved over 5 minutes using the ExionLC AD UHPLC system from Applied Biosystems/MDS SCIEX (Foster City, CA, USA). A Kinetex® EVO C18 column (100×2.1 mm internal diameter, 5 μm) with column oven temperature of 30° C., injection volume of 2 μL, and flow rate of 0.2 mL/min were used. An isocratic mobile phase consisting of 0.1% formic acid (60%) and 0.1% formic acid in acetonitrile (40%) was used. An API 5500 QTrap Triple-Quadrupole mass spectrometer with a TurbolonSpray™ source from Applied Biosystems/MDS SCIEX was used to accomplish mass spectrophotometric detection. The multiple reactions monitoring (MRM) method was operated in positive-ion mode to determine the best transition pairs for zileuton (m/z 237.3-161.1) and zileuton-d4 (m/z 241.2→165.1), respectively. The Analyst® 1.6.3 software (Foster City, CA, USA) was used to quantify analyte and internal standard concentrations. (Additional assay performance parameters and are shown in Appendices 1-5, for reviewing purposes only).

Preparation of standards and samples for quantification. Working solutions of zileuton in 2× concentration dilutions from 0.1875 mg/L to 96 mg/L were prepared in 40% methanol, from a 10 mg/mL stock solution in ethanol stored at −20° C. The internal standard solution was prepared by dilution of a stock solution to 20 mg/L. Following preparation of the working solutions, 15 μL of serum was mixed with 5 μL zileuton working solution and 10 μL zileuton-d4 working solution. Rat serum samples (15 L) were mixed with 10 μL of internal standard and 5 μL 40% methanol. Then 300 μL of cold acetonitrile was added, and the mixture was vortexed for 30 seconds followed by centrifugation at 15,000×G for 15 minutes. The supernatant was recovered and dried under a stream of nitrogen. Standards and samples were then reconstituted in 600 μL LCMS-grade water and centrifuged for 15 minutes at 15,000×G. The linear range of the assay was 0.125-32 mg/L and the LLOQ was 0.0625 mg/L. Each calibration curve was calculated using a 1/x weighting of the linear regression. Intra- and interday precision and accuracy were acceptable (CV % and error %<10%). The percent recovery was very good (>85%) and the matrix effect was not significant (<±10%).

### Example 4: Selective Kidney Accumulation of Nanoparticles in Kidney of Mice without Toxicity

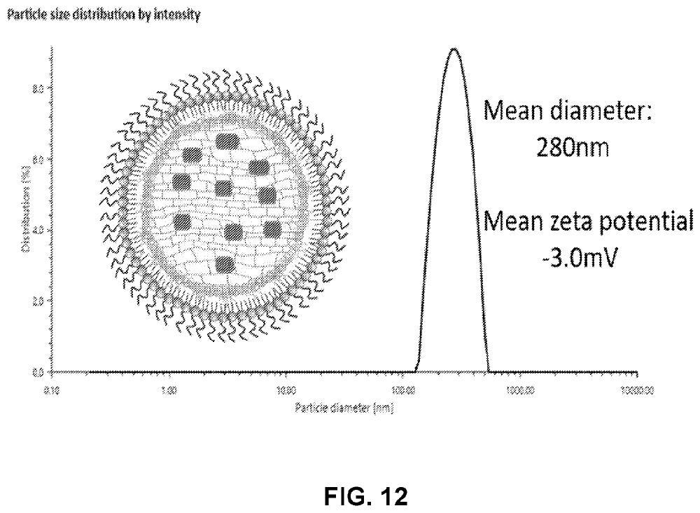

A lipid-polymer hybrid polyplex nanoparticles (NPs) composed of polysaccharide, pegylated lipids, and phosphate counter ions was developed, as schematically depicted in FIG. 12. The NPs have a mean hydrodynamic diameter of 280 nm and near neutral charges (−3 mV). The NPs were designed to have a bigger colloidal particles size, so that NPs are not subject to kidney filtration into the urine (molecules with a diameter smaller than 10 nm fall below the kidney filtration threshold and can pass through the glomerulus and be excreted into urine quickly). The NP compositions used herein are biomaterials known to be biodegradable in vivo into biologically benign components.

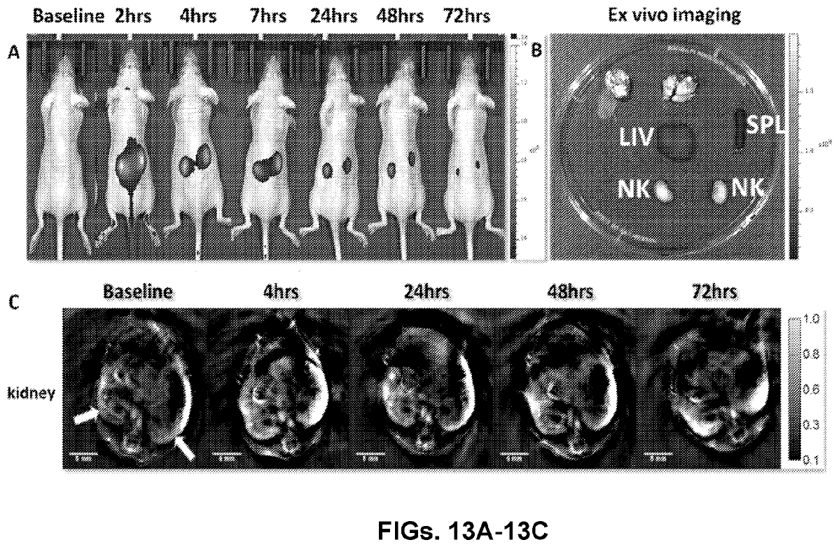

A near infrared fluorescent dye Alexa Fluor 750 (AF750) was conjugated to the NPs and in vivo whole-body imaging was performed in normal nude mice after intravenous (IV) administration. Fluorescence intensity was monitored for up to 3 days. The free unconjugated AF750 control were quickly cleared out of the mice within a few hours (data not shown). On the other hand, as shown in FIG. 13A, the real-time whole animal imaging data showed that kidneys displayed the highest accumulation of AF750-labeled NP after two (2) hours post-administration, and remained visualized after 2-3 days without apparent toxicity. As shown in FIG. 13B, ex vivo imaging showed more NP accumulation in the kidneys than the liver or the spleen three (3) days post NP injection. As shown in FIG. 13C, multispectral optoacoustic tomography (MOST) imaging also demonstrated strong kidney accumulation of NPs. These data demonstrate that the NPs have unique kidney-homing ability when injected in vivo.

### Example 5: Tissue and Cellular Localization of Nanoparticles in Proximal Tubule Epithelium of the Kidney

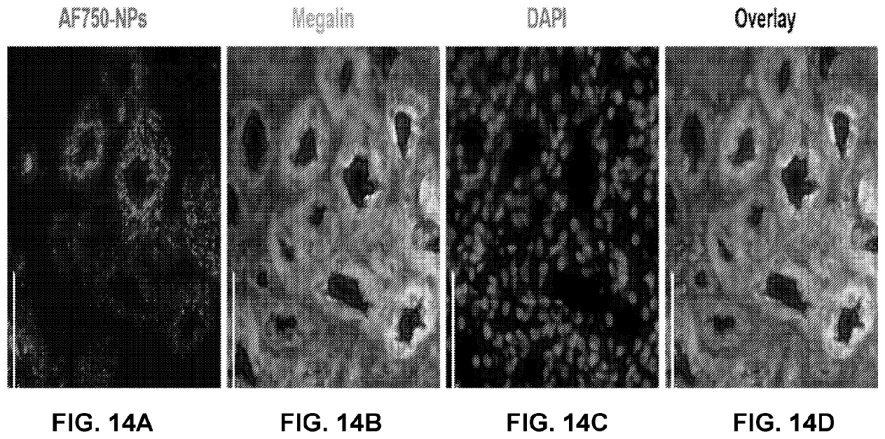

Three (3) days after an IV injection of AF750-labeled NPs as described in Example 2, anesthetized mice were systemically perfused with PBS buffer. Mice were euthanized and kidneys harvested. Frozen kidneys were sectioned to allow examination of NP localization. FIGS. 14A-14D depict fluorescence images (40×) of kidney after 3 days post IV administration of AF750-labeled NPs in mice. Kidneys were stained with anti-mouse megalin antibody and cell nuclei was stained with DAPI (Red: NPs (FIG. 14A). Green: megalin (FIG. 14B), Blue: nuclei (FIG. 14A), and all overlaid in FIG. 14D) (n=3). As shown in FIGS. 14A-14D, when the tissue sections were counterstained with DAPI, the NPs (red) were found to largely accumulate in the kidneys. The NPs (red) were visualized mainly in close proximity of the megalin-positive renal tubular epithelial cells (green), indicating that the NPs are likely endocytosed into renal tubules via megalin receptor.

### Example 6: Optimization of Nanoparticles for Targeted Delivery to the Site of Injury

To enhance delivery of a drug of interest (e.g., zileuton) to the site of injury, nanoparticles using lipids and polysaccharides as major components, as shown in FIG. 12, are developed and optimized. The materials composing the nanoparticles are known to be biocompatible and biodegradable in vivo. Cargo (i.e., zileuton) is incorporated in the inner cavity of the nanoparticles. Alternatively, cargo can be conjugated externally to the nanoparticles. In some cases, modified chitosan is incorporated, which can form nanoparticles by interacting with the ionic cross-linking phosphate salt derivatives via the ionic gelation method (Koukaras et al. 2012 Mol Pharm 9:2856-62; Sacco et al. 2016 Int J Biol Macromol 92:476-83). The amphoteric lipids are further added to generate more compact hybrid nanoparticles via an electrospraying technique. Pharmacokinetic investigations similar to those described in Example 2 are undertaken with various zileuton-nanoparticle formulation(s) including the hybrid nanoparticles described in Example 3 (having a mean hydrodynamic diameter of 280 nm and near neutral charges). An increase in zileuton area under the concentration-time curve (AUC₂₄) in renal tissue of >50% is considered a desirable delivery.

The interaction of NPs with the kidney is size-dependent, owing to the unique ultrastructure of kidney anatomy at the micro- and nano-scales. If the nanoparticles are too small (<6 nm), they can easily cross the glomerular filtration barriers and be excreted in the urine. On the other hand, if the nanoparticles are too big (˜500 nm), they can accumulate and are cleared by organs of the reticuloendothelial system (RES). Fine tuning the size of nanoparticles to allow targeting of nanoparticles in the kidneys, while minimizing exposure in other organs is critical to use zileuton nanoparticles as a renal protectant. The ratio and concentration of the polysaccharide, lipids, and counter ions are optimized to generate various nanoparticles with a size range of 50-250 nm. Their kidney tubular accumulations are evaluated. Based on results of a systematic investigation of the charge effect of NPs in kidney accumulation generate optimal NPs with high kidney accumulation without toxicity are generated. Dynamic light scattering (DLS) and nanoparticle tracking analysis (NTA) techniques are utilized to determine the size as well as zeta potential of the NPs. In vivo imaging method described in FIGS. 13A-13C and Example 2 is used to determine the optimal size and surface charge of NPs that render the highest kidney accumulation with the lowest RES uptake. Additionally, the standard endotoxin contamination assay, microbial contamination assay, in vitro hemolysis assay, in vitro complement activation assay, and stability assay are conducted to evaluate therapeutic effects of NP-zileuton formulation.

Embodiments include a pharmaceutical composition comprising a therapeutically effective amount of zileuton and a biologically safe excipient formulated for parenteral administration. In certain examples, the excipient contains water, saline, polyethylene glycol (PEG), propylene glycol, glycerin, ethanol, sorbitol, triacetin, polyoxyethylated glycerides, polyoxyethylated oleic glycerides, hyroxypropyl-beta-cyclodextrin, a surfactant, or any combination thereof. In certain examples, the PEG has molecular weight of about 350-650 Daltons, such as PEG 400, PEG 500, PEG 600. In certain examples, the surfactant includes one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient contains ethanol, PEG 400, and water. In certain examples, the excipient contains ethanol, PEG 400, and water in 10%:40%:50% in volume. In certain examples, the excipient contains ethanol, PEG 400, and water in 10%:25%:65% in volume. In certain examples, the excipient includes a nanoparticle comprising a polysaccharide, a pegylated lipid, phosphate counter ions, or a combination of any thereof. In certain examples, the nanoparticle has a mean hydrodynamic diameter of about 50-300 nm and near neutral charge. In certain examples, the nanoparticle has a mean hydrodynamic diameter of about 280 nm and a mean zeta potential of about −3 mV. In certain examples, the nanoparticle contains zileuton in an inner cavity thereof.

In certain examples, the pharmaceutical composition comprises at least about 1 mg/ml of zileuton. In certain examples, the zileuton degrades less than 10% over 142 hours at −20° C., 4° C., or 22° C. in the pharmaceutical composition.

Embodiments include the pharmaceutical composition provided herein, for use in preventing, delaying onset of, or treating acute kidney injury or nephrotoxicity in a subject. In certain examples, the AKI or nephrotoxicity is induced by an antibiotic or an anti-cancer drug. In certain examples, the antibiotic is one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug is one or more of ifosfamide, ipilimumab, pembrolizumab and nivolumab.

Embodiments include the pharmaceutical composition provided herein, for intravenous, intraperitoneal, subcutaneous, intramuscular, or transdermal administration to a subject. Embodiments include the pharmaceutical composition provided herein, for parenteral administration to a critically ill subject or a subject having severe infection or infection with multidrug resistant bacteria.

Embodiments include methods for preparing a pharmaceutical composition by combining a therapeutically effective amount of zileuton and a biologically safe excipient for parenteral administration. In certain examples, the excipient includes two or more of water, saline, polyethylene glycol (PEG) (such as PEG of molecular weight of about 350-650 Daltons, PEG 400, PEG 500, PEG 600), propylene glycol, glycerin, ethanol, sorbitol, triacetin, polyoxyethylated glycerides, polyoxyethylated oleic glycerides, hyroxypropyl-beta-cyclodextrin, a surfactant, or any combination thereof. In certain examples, the surfactant can be one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient contains ethanol, PEG 400, and water. In certain examples, the excipient contains ethanol, PEG 400, and water in 10%:40%:50% in volume. In certain examples, the excipient contains ethanol, PEG 400, and water in 10%:25%:65% in volume.

In certain examples, the excipient contains a nanoparticle with a polysaccharide, a pegylated lipid, phosphate counter ions, or a combination of any thereof. In certain examples, the nanoparticle has a mean hydrodynamic diameter from about 50 nm to about 300 nm of about 50-300 nm and near neutral charge. In certain examples, the nanoparticle has a mean hydrodynamic diameter of about 280 nm and a mean zeta potential of about −3 mV.

In certain examples, the method includes loading the zileuton into an inner cavity of the nanoparticle, thereby preparing the pharmaceutical composition for parenteral administration. In certain examples, the pharmaceutical composition comprises at least about 1 mg/ml of zileuton. In certain examples, the zileuton degrades less than 10% at −20° C., 4° C., or 22° C. in the pharmaceutical composition.

Embodiments include a method of preparing the pharmaceutical composition provided herein, for use in preventing, delaying onset of, or treating acute kidney injury or nephrotoxicity in a subject. In certain examples, the AKI or nephrotoxicity is induced by an antibiotic or an anti-cancer drug. In certain examples, the antibiotic is one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug is one or more of ifosfamide, ipilimumab, pembrolizumab and nivolumab.

Embodiments include a method of preparing the nanoparticle composition for intravenous, intraperitoneal, subcutaneous, intramuscular, or transdermal administration to a subject. Embodiments include a method of preparing the nanoparticle composition for parenteral administration to a critically ill subject or a subject having a severe infection or infection with multidrug resistant bacteria. Embodiments include a method of administering a therapeutically effective amount of zileuton to a subject by administering parenthetically to the subject a nanoparticle composition comprising a therapeutically effective amount of zileuton and a biologically safe excipient formulated for parenteral administration. In certain examples, the method includes administering the nanoparticle composition intravenously, intraperitoneally, subcutaneously, intramuscularly, or transdermally to the subject.

In certain examples, the method includes administering the pharmaceutical composition in a single dose to the subject. In certain examples, the method includes administering the pharmaceutical composition in multiple doses, in regular or varied interval to the subject. In certain examples, the method includes administering the pharmaceutical composition to achieve serum zileuton concentration of about 1 mg/kg to about 12 mg/kg in the subject. In certain examples, the method includes administering about 880 mg to about 3000 mg zileuton to the subject. In certain examples, the method includes administering the pharmaceutical composition to a critically ill subject or a subject having a severe infection or infection with multidrug resistant bacteria. In certain examples, the method prevents, delays onset of, or treats acute kidney injury or nephrotoxicity in a subject. In certain examples, the AKI or nephrotoxicity is induced by an antibiotic or an anti-cancer drug.

Embodiments include a method of treating a subject with infection, comprising administering parenterally to the subject a pharmaceutical composition comprising (i) a therapeutically effective amount of zileuton and a biologically safe excipient formulated for parenteral administration; and (ii) a therapeutically effective amount of a nephrotoxicity-inducing antibiotic selected from the group consisting of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4.

Embodiments include a method of treating a subject with cancer, comprising administering parenterally to the subject a pharmaceutical composition comprising (i) a therapeutically effective amount of zileuton and a biologically safe excipient formulated for parenteral administration; and (ii) a therapeutically effective amount of a nephrotoxicity-inducing anticancer drug selected from the group consisting of ifosfamide, ipilimumab, pembrolizumab, and nivolumab. In certain examples, the therapeutically effective amount of zileuton ranges from about 0.5 mg/kg to 200 mg/kg. In certain examples, the therapeutically effective amount of zileuton ranges from about 1 mg/kg to 12 mg/kg. In certain examples, the method includes administering the pharmaceutical composition comprising about 880 mg to about 3000 mg zileuton to the subject.

Embodiments include a kit for parenteral administration of zileuton, including a pharmaceutical composition comprising a therapeutically effective amount of zileuton and a biologically safe excipient formulated for parenteral administration, and an instruction for use.

In certain examples, the excipient includes water, saline, polyethylene glycol (PEG), PEG with molecular weight of about 350-650 Daltons, PEG 400, PEG 500, PEG 600, propylene glycol, glycerin, ethanol, sorbitol, triacetin, polyoxyethylated glycerides, polyoxyethylated oleic glycerides, hyroxypropyl-beta-cyclodextrin, a surfactant, or any combination thereof. In certain examples, the surfactant can be one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient includes ethanol, PEG 400, and water. In certain examples, the excipient contains ethanol, PEG 400, and water in 10%:40%:50% in volume. In certain examples, the excipient contains ethanol, PEG 400, and water in 10%:25%:65% in volume. In certain examples, the excipient includes a nanoparticle comprising a polysaccharide, a pegylated lipid, phosphate counter ions, or a combination of any thereof. In certain examples, the nanoparticle has a mean hydrodynamic diameter of about 50-300 nm and near neutral charge. In certain examples, the nanoparticle has a mean hydrodynamic diameter of about 280 nm and a mean zeta potential of about −3 mV. In certain examples, the nanoparticle contains zileuton in an inner cavity thereof. In certain examples, the pharmaceutical composition includes at least about 1 mg/ml of zileuton. In certain examples, the zileuton degrades less than 10% over 140-150 hours at −20° C., 4° C., or 22° C. in the pharmaceutical composition. In certain examples, the kit is for preventing, delaying onset of, or treating acute kidney injury or nephrotoxicity in a subject. In certain examples, the AKI or nephrotoxicity is induced by an antibiotic or an anti-cancer drug. In certain examples, the kit contains an antibiotic, which is one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the kit contains the anti-cancer drug, which is one or more of ifosfamide, ipilimumab, pembrolizumab and nivolumab. In certain examples, the kit contains the pharmaceutical composition for intravenous, intraperitoneal, subcutaneous, intramuscular, or transdermal administration to a subject. In certain examples, the kit is for parenteral administration of zileuton to a critically ill subject or a subject having severe infection or infection with multidrug resistant bacteria.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A pharmaceutical composition comprising a therapeutically effective amount of zileuton and a biologically safe excipient formulated for parenteral administration.

2. The pharmaceutical composition of claim 1, wherein the excipient comprises water, saline, polyethylene glycol (PEG) of molecular weight ranging from about 350-650 Daltons, propylene glycol, glycerin, ethanol, sorbitol, triacetin, polyoxyethylated glycerides, polyoxyethylated oleic glycerides, hyroxypropyl-beta-cyclodextrin, a surfactant, or any combination thereof.

3. The pharmaceutical composition of claim 2, wherein the surfactant comprises one or more of hydroxypropylcellulose 20, polysorbate 80, polysorbate 20, sorbitan monooleate NF, polyoxyl 40 hydrogenated castor oil, and polyoxyl hydroxystearates.

4. The pharmaceutical composition of claim 2, wherein the excipient comprises ethanol, PEG 400, and water.

5. The pharmaceutical composition of claim 4, wherein the excipient contains ethanol, PEG 400, and water in 10%:40%:50% by volume.

6. The pharmaceutical composition of claim 4, wherein the excipient contains ethanol, PEG 400, and water in 10%:25%:65% by volume.

7. The pharmaceutical composition of claim 1, wherein the excipient comprises a nanoparticle comprising a polysaccharide, a pegylated lipid, phosphate counter ions, or any combination thereof.

8. The pharmaceutical composition of claim 7, wherein the nanoparticle has a mean hydrodynamic diameter of about 50-300 nm and near neutral charge.

9. The pharmaceutical composition of claim 7, wherein the nanoparticle contains zileuton in an inner cavity thereof.

10. The pharmaceutical composition of claim 1, comprising at least about 1 mg/ml of zileuton.

11. The pharmaceutical composition of claim 1, further comprising an antibiotic or an anti-cancer drug.

12. A method of preventing, delaying onset of, or treating acute kidney injury (AKI) or nephrotoxicity in a subject by administering the composition of claim 1.

13. The method of claim 12, wherein the AKI or nephrotoxicity is induced by an antibiotic or an anti-cancer drug.

14. The method of claim 13, wherein the antibiotic is one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4.

15. The method of claim 13, wherein the anti-cancer drug is one or more of ifosfamide, ipilimumab, pembrolizumab and nivolumab.
